(19) United States
(12) Patent Application Publication
(10) Pub. No.: US 2003/0233034 A1
(43) Pub. Date: Dec. 18, 2003

(54) Apparatus for measuring vital functions

(76) Inventors: Alpo Varri, Tampere (FI); Mikko Koivuluoma, Lampaala (FI); Jarmo Alametsa, Tampere (FI); Ulla Ruotsalainen, Helsinki (FI)

Correspondence Address: SWIDLER BERLIN SHEREFF FRIEDMAN, LLP, 3000 K STREET, NW, BOX IP, WASHINGTON, DC, 20007, US

(21) Appl. No.: 10/460,317

(22) Filed: Jun. 13, 2003

(30) Foreign Application Priority Data
Jun. 13, 2002 (FI) 20021145

(51) Int. Cl. A61B 5/00

(52) U.S. Cl. 600/301

(57) ABSTRACT

The invention relates to an apparatus for measuring the vital functions of a patient, which apparatus comprises at least a measuring chair (20) or a corresponding means suitable for sitting, which measuring chair further comprises one or more measuring sensors (2, 3, 6, 7) for measuring one or more vital functions of the patient sitting in the measuring chair, in a non-invasive manner from the outside of the patient's body. According to the invention, said one or more measuring sensors (2, 3, 6, 7) are placed in the structures of the measuring chair (20) in a substantially unnoticeable way, and the measuring chair (20) is preferably designed to resemble an ordinary chair intended for non-medical use, or a corresponding furniture-like means suitable for sitting. By means of the invention, it is possible to reduce the distortion caused by fear of doctors in the measurement results. The invention is particularly suitable, for example, for ballistocardiographic measurements and/or measurements relating to pulmonary functions.

The invention relates to an apparatus for measuring vital functions according to the preamble of claim 1.

In the following, a person subject to measurements and examination will be called briefly and generally a patient. However, it should be noted that this term is not intended to limit the invention solely to diagnostics relating to the treatment of a disease of said person but, as it will be disclosed below, also other measurements and investigations relating to the vital functions of the person are widely feasible. Consequently, the measurements and examinations to be taken by the apparatus according to the invention can also be used to evaluate, for example, the physical condition of a completely healthy person.

In practice, the vital functions of the person under examination, i.e. the patient, are typically measured by attaching one or more measuring sensors to the patent in a sitting or lying position. By the measuring sensors, the vital function of the patient to be examined, which may be an electrical, physical or another phenomenon, is converted to such a form that makes it possible to analyse and diagnose said vital function. In practice, however, the measuring of the vital functions by means of sensors attached to the patient is normally applicable to such measurements only, which also involve, in addition to the patient, a person to take the measurement. The attachment of the measuring sensors to the patient may also require particular expertise which, for example, a general practitioner does not always necessarily have. Furthermore, the attachment of the measuring sensors to the patient requires time and, in most cases, at least partial undressing of the patient.

For the above-mentioned reasons, the vital functions of the patient are normally measured at laboratories of various health care organizations with personnel specialized in the use of the necessary electrophysiological measuring instruments. It is a very well known fact as such that the person coming to the practice of a doctor or another health care professional may be nervous because of the examination he/she should undergo. In this case, the “white coat syndrome”, or fear of doctors, is a common concept. When the patient is nervous before the measuring situation, in which, for example, sensors or the like are attached to his/her body by another person, the measurement result relating to the vital functions of the patient may differ even to a great extent from a normal situation in which the patient would be physically and mentally relaxed. Examples of such vital functions which may be easily affected by the tension of the patient include, for example, the patient's pulse rate and blood pressure. Consequently, in situations of the above-mentioned type, the distortion in the measurement result is caused explicitly by the tension of the patient in the measuring situation. The measuring instruments used in the measurement are, as such, exact and give good results if the patient could be relaxed in the measuring situation.

From prior art, solutions are also known, in which devices for measuring vital functions are combined with various chairs or the like, wherein, in certain situations, it is also possible that the patient him/herself can take measurements on him/herself. When measurements are taken, for example, at home, it is possible to reduce the anxiety of the patient in connection with the measuring situation and thereby to improve the reliability of the measurement results in this respect. Furthermore, it is easier to take the measurements at regular intervals and more frequently.

Japanese patent publication JP 03007136 A discloses a solution, in which an armrest for measuring vital functions is connected to a toilet seat. The armrest comprises a measuring means inside which the patient places the middle finger of his/her left hand to measure the blood pressure and the pulse rate. To take the measurement, the patient must place his/her hand in a correct predetermined manner in the measuring means placed on the armrest of the toilet seat.

In U.S. Pat. Nos. 5,544,649 and 5,441,047, vital functions of a patient are measured in a chair whose armrest is equipped with an auxiliary device for taking the measurements. The auxiliary device comprises, for example, a cuff to be tightened around the arm to measure the patient's blood pressure and pulse rate. Furthermore, measuring sensors may be attached to the patient's chest for other measurements relating to the electrical function of the heart. So that the measurement results would be reliable, the patient must know how to fix the cuff and possible other measuring sensors in the correct way. When the patient takes measurements at home, he/she can simultaneously communicate visually for consultation with a health care professional via a suitable telecommunications network. The measurement results can also be transmitted in electrical format to the health care organization.

In the patent publication WO 01/87143, the idea of placing sensors for measuring the vital functions of a patient in a chair is developed further. In this arrangement, the chair as such is specially made and is equipped with several sensors to be attached to the patient. Furthermore, the chair, as such, comprises weight sensors for determining the patient's weight. Also in this case, the use of the chair and the attachment of the sensors to the patient requires certain know-how to secure reliable measurement results.

From prior art, so-called ballistocardiographic measurements are also known for the measurement of force effects by the cardiac function on the body. When the heart pumps blood into the aorta and the pulmonary artery, the body is subjected to a recoil force in the direction opposite to the blood flow, the magnitude and the direction of the recoil force changing with the functional phase of the heart. This recoil force can be measured by sensitive force sensors outside the body, wherein the measurement can be made in a fully non-invasive manner. Conventionally, ballistocardiographic measurements have been taken when the patient is lying on a bed supported by force sensors.

From prior art, arrangements are also known in which the force sensors intended for ballistocardiographic measurements are placed in a special measuring chair. The laboratory of Applied Electronics Studies at Helsinki University of Technology has published, on its website (http://www.hut.fi/Units/Electronics/), an arrangement for a ballistocardiographic measuring chair. On said website, an arrangement for a measuring chair is presented in the publications Ballistocardiography—New Methods for Measuring, Lasse Leppäkorpi and Suvi Koskinen (included as an appendix to this application) and Ballistocardiography—A New Method for Heart Monitoring, Jarmo Ritola, Tuija Vächä-Rahka and Raimo Sepponen (included as an appendix to this application). Said measuring chair is a special chair with a massive structure and appearance, and it is intended particularly for use in hospitals or laboratories.

All the above-presented arrangements of prior art share the common feature that when taking the measurement, the patient is always very aware of being subjected to measuring operations. The taking of the measurements will always require either that sensors or cuffs are attached to the patient's body, or at least that the patient sits in a measuring chair with a special structure which is easily recognized as a medical apparatus because of its appearance and presence. These facts will easily make the patient feel some kind of anxiousness, or fear of doctors, in the measuring situation, and distortions are thus caused in the patient's measurement results because his/her body is in a state of stress. It is known as such that even a slight tension will cause significant changes, for example, in the pulse level and the blood pressure of even a healthy person.

The present invention relates to a measuring apparatus and particularly a measuring chair, by means of which the above-mentioned drawbacks of the arrangements of prior art can be avoided. The measuring chair according to the invention is primarily characterized in that the structure of the measuring chair is designed so that the chair has the external appearance of an ordinary chair and no sensors or the like need to be attached to the patient to take the measurements. Thus, when the patient sits in the measuring chair, the probability that he/she will feel anxious because of the measuring situation is significantly smaller than in a situation in which the patient must sit in a special chair with a very technical appearance which may invoke a mental picture of, for example, a dentist's chair or the like.

By means of the apparatus and measuring chair according to the invention, the patient's vital functions can be measured, if necessary, also in such a way that the person is not at all aware of being subjected to measuring. This will considerably expand the applications of the invention beyond conventional uses.

To put it more precisely, the apparatus according to the invention is primarily characterized in what will be presented in the characterizing part of the appended independent claim 1. The other, dependent claims will present some preferred embodiments of the invention.

In an advantageous embodiment of the invention, the measuring sensors needed for the measurements are placed in the actual seat part, backrest and/or armrests of the measuring chair, underneath the surface material of the chair, in such a way that they are externally imperceptible. Preferably, the sensors are implemented with an electro-mechanical film (EMFi), wherein the sensors can be made very sensitive and with a flat structure, wherein they can be easily installed to be unnoticeable in the structures of the chair. Also the other electronic devices or the like, needed for the transmission or processing of the measuring signals, are preferably placed unnoticeably underneath or inside the structures of the measuring chair. Preferably, the measuring signals are transmitted to the other devices needed for the processing, analyzing, storage or displaying of the measuring signals in a wireless manner, for example by using the Bluetooth technology. Other possible wireless data transmission techniques suitable for the purpose include, for example, the WLAN and ZigBee technologies presented in the IEEE 802.11 and IEEE 802.15 standards, HomeRF, Wireless 1394, and the like.

The apparatus and measuring chair according to the invention can be used for measuring weak force effects and pressure vibrations relating to the patient's lung function, heart function, blood circulation, body tremor, and the function of the digestive system, which are conveyed via the patient's body to the measuring sensors in the chair. By analyzing these signals, it is possible to determine various aspects relating to the patient's body condition. The invention is particularly suitable for so-called ballistocardiographic measurements.

When said measurements are taken differentially by separating the common-mode interference signal, measured by reference sensors or the like, from the actual measurement signal, the sensitivity and interference tolerance of the measurements can be significantly improved.

Using the measuring chair according to the invention, it is possible to take measurements in the facilities of a health care organization or the like, as well as at the patient's home. The measuring chair can also be made as an automatic apparatus suitable for entertainment use or, for example, use at pharmacies, where the apparatus measures the user's physical condition for a charge.

The invention has the advantage that the vital functions of the patient can be measured without a need for the patient to undress in any way. Consequently, the measuring chair can be used to measure the vital functions of the patient, unbeknown to the patient, for example in the waiting room outside the reception room at a health clinic, wherein the measurement corresponds better to the patient's normal condition. To take the measurements, the patient must only sit down normally in the chair, substantially in the same way as if sitting in an ordinary chair.

The advantageous embodiments of the invention and their advantages to the prior art will become clear for a man skilled in the art from the following detailed description of the invention. In the description, reference will be made to the appended drawings, in which:

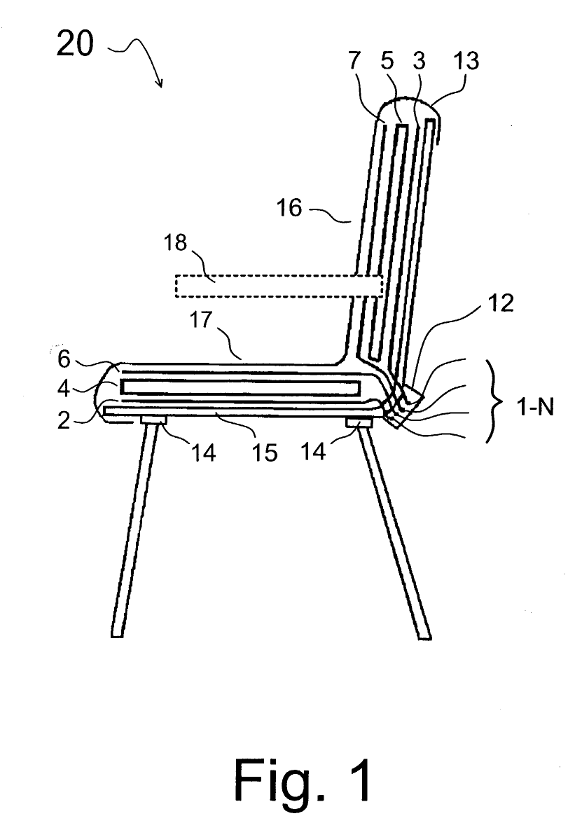

FIG. 1 shows, in principle, an embodiment of the measuring chair according to the invention,

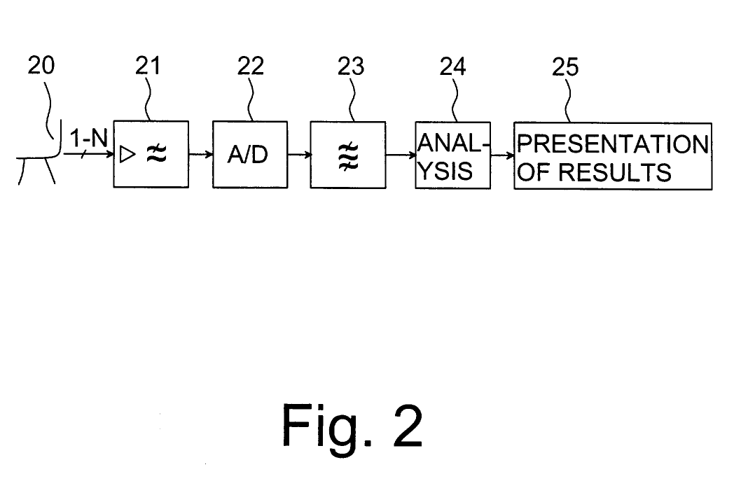

FIG. 2 shows, in a principle block chart, a way of processing measurement signals according to the invention,

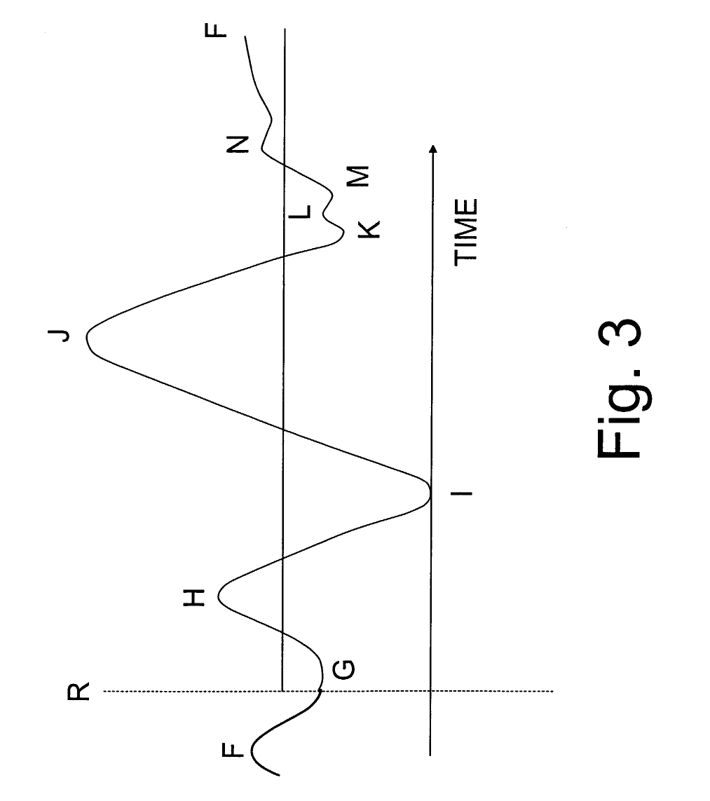

FIG. 3 shows, in principle, the dominant mode of a ballistocardiographic signal, and

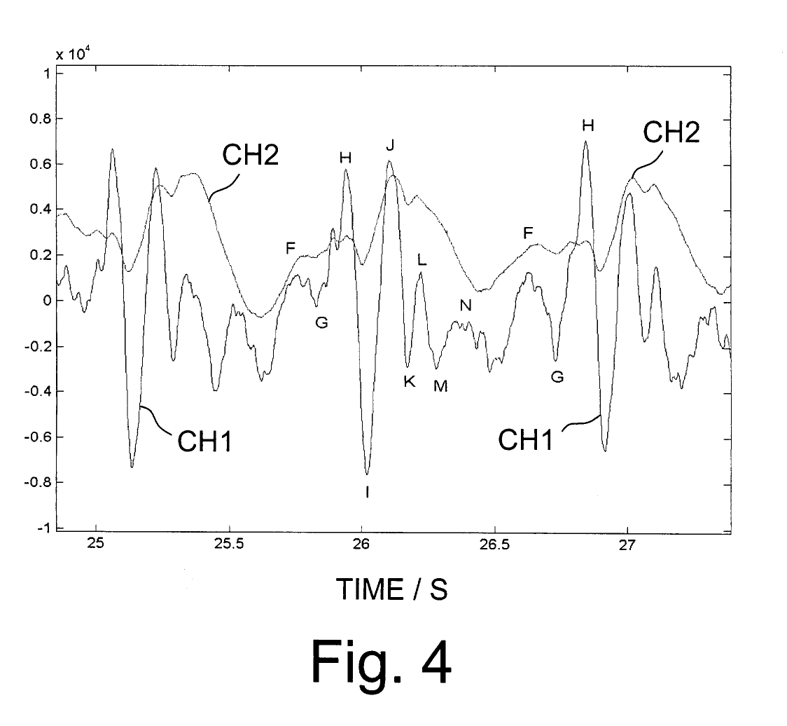

FIG. 4 shows examples of ballistocardiographic signals measured of a testee.

FIG. 1 shows, in principle, an advantageous embodiment of a measurement chair 20 according to the invention.

The frame 15 of a chair, ordinary as such, is coated with one or more flat first measuring sensors 2, 3, which first measuring sensors can be placed in the seat part 17 and/or in the backrest 16 of the chair. The first measuring sensors 2, 3 are coated with cushions 4, 5. The cushions 4, 5, in turn, are coated with one or more flat second measuring sensors 6, 7. Both the first measuring sensors 2, 3 under the cushion and the second measuring sensors 6, 7 on the cushion are connected to conducting wires, by means of which a signal 1−N recorded by the sensors can be led via a coupling 12 to a device for amplifying, storing, analyzing, or displaying the signal numerically or graphically.

Instead of the coupling 12, the signals 1−N can also be led, for example, to the lower surface of the seat part or of the frame of the chair, where a compact electronic device or the like is placed (not shown in FIG. 1,), encapsulated to be unnoticeable and used for processing the signals in a suitable way and for transmitting the measurement data in a wireless manner to a device making an analysis, for example a computer. The method of transmission can be analog or digital. Short-range wireless transmission can be implemented, for example, by using the Bluetooth technology.

According to the invention, the measuring chair 20 is provided with normal surface upholstery 13. The upholstery 13 can be made of, for example, fabric or plastic, and it may preferably also comprise a conductive layer, wherein the upholstery 13 simultaneously forms a Faraday cage to attenuate external electromagnetic fields. The conductive layer can be formed, for example, of a wire mesh, of a thin metal film or, for example, by treating the inner surface of the upholstery fabric 13 with a graphite spray or the like.

The seat part 17 and the backrest 16 of the measuring chair 20 can each be provided with several measuring sensors. If necessary, measuring sensors can also be placed in armrests 18 of the chair, shown with a broken line in FIG. 1.

The implementation of the measuring sensors in two layers in such a way that there is a layer (cushion 4, 5) for attenuating vibrations between the first lower measuring sensors 2, 3 and the second upper measuring sensors 6, 7, is advantageous because when the signal of the lower measuring sensor 2, 3 is thus subtracted, for example, by adaptive signal processing, from the signal of the upper measurement sensor 6, 7, a more clear signal is left, originating from the vital functions of the patient. By means of the lower measuring sensor 2, 3, it is possible to eliminate from the signal common-mode interference which is caused, for example, by the movements of the patient when he/she changes his/her position in the measuring chair. The signals from the cardiac or pulmonary functions are transmitted stronger in the upper second measuring sensors 6, 7 which are placed closer to the patient's body and there are no cushions 4, 5 attenuating the vibrations. By means of the lower measuring sensors 2, 3, for example vibration interference transmitted via the floor to the structures of the measuring chair 20 can also be subtracted from the signal.

In addition to said measuring sensors 2, 3, 6, 7, the measuring chair 20 can also be provided with one or reference sensors 14 for recording vibrations in the measuring environment, such as vibrations transmitted via the floor to the chair. In FIG. 1, the measuring sensors 14 are placed in connection with the legs of the chair, but the reference sensors can also be placed in another way. For the sake of clarity, the wiring of the reference sensors is not illustrated in FIG. 1. The effect of interference can be compensated for by subtracting the signal given by the reference sensors 14 from the signals of the actual measuring sensors 2, 3, 6, 7.

It is typical of the invention that all the above-mentioned measuring and reference sensors placed in the measuring chair 20 are placed in the structure of the chair in an unnoticeable way so that the seated person does not get an idea that the chair is a particular medical apparatus.

In one embodiment of the invention, measuring sensors are placed in the seat part 17 or in the backrest 16 of the chair in such a way that separate sensors are provided for the right and left sides of the body, wherein information can be recorded from the right and left sides of the patient's body separately. In the seat part of the measuring chair 20, the measuring sensors 2, 6 can be divided, for example, in two separate parts which are placed underneath the patient's right and left buttocks separately. Signals from different parts of the patient's body can also be recorded by measuring sensors placed in the armrests 18.

The flat measuring sensors 2, 3, 6, 7 can be implemented, for example, by using an electromechanical film (EMFi), whose structure and function is described in more detail, for example, in U.S. Pat. No. 4,654,546. The advantage of the electromechanical film is that it can be used to implement, at reasonable costs, a very sensitive and flat film-like sensor which converts the force on the film directly to an electrical value. Suitable measuring sensors also include other sufficiently compact sensors for measuring weight/force, as long as their sensitivity is sufficient, i.e., they are sufficiently sensitive to detect, for example, body movements caused by a heartbeat. Consequently, for example capacitive or piezoelectric force sensors can thus be used as the sensors.

In addition to the measuring sensors 2, 3, 6, 7 measuring the vital functions, it is possible to use, if necessary, for example a sensor or sensors measuring the patient's absolute weight, because for example in ballistocardiographic measurements, the patient's weight has been shown to have a slight effect on the measurement result.

The signals obtained from from the measuring sensors 2, 3, 6, 7, 14 are processed in a way shown in the principle block chart of FIG. 2. The functions of said block chart can be partly placed in an electronic unit or the like placed unnoticeably in the structures of the measuring chair 20, or alternatively, the functions can be implemented in a separate device, for example a computer or another signal processing device.

It will be obvious for a man skilled in the art that the signal obtained from the measuring sensors 2, 3, 6, 7, 14 can also be processed by analog electronics only, but it is most advantageous to convert the signal to digital format wherein there are more possibilities for its processing and the analyzing method can be changed, if necessary, for example by modifying the software of the device.

In FIG. 2, the signals produced by the sensors 2, 3, 6, 7, 14 of the measuring chair 20 are indicated with the references from 1 to N. At first, the signals 1−N are suitably amplified in a preamplifier 21, and simultaneously they can be filtered by low-pass filtering for too high frequencies, for a possible DC component, and/or for other interference. Next, the signals are preferably converted to digital format by an analog-to-digital converter 22. In the digital format, the signals are processed further and they are filtered by digital filtering 23 of frequency components unnecessary for the analysis. At this stage, it is also preferable to reduce external interference from the signals representing the patient's body functions. The suppression of interference can also be implemented in the preamplifier 21. At an analysis step 24, features representing the condition of vital functions, known as such, are searched for in the signal by various signal processing methods in the time and frequency domains. It is also possible to compare the measured results either with results recorded previously from the same person or with reference material collected from other persons, to find deviations.

After the processing and analysis of the signals 1−N in the above-described manner, the results can be presented in a suitable way (block 25). The measurement results and the analysis made on the basis of them and representing the patient's condition can also be stored, for later use, in a memory device, such as, for example, fixed disks, diskettes, compact discs and digital versatile discs with their variations, memory cards, magnetic tapes, etc. Of the information, it is possible to store not only the original measurement signals but also various intermediate results or the final result of the analysis.

Preferably, the measurement signals 1−N are processed in a suitable computer-based device, such as a personal computer. When the measuring signals 1−N are transferred in a wireless manner from the measuring chair 20 to the device for processing and analysis of the measuring signals, there is no need for conductors or the like which should be coupled to the measuring chair 20 and would reveal that the chair is a medical apparatus. The electronics and, for example, the equipment required for the wireless communication, to be possibly placed in the measuring chair 20, can be implemented by battery operation, wherein no conducting wires need to be coupled to the measuring chair 20 during its actual use.

When the patient to be examined sits down in the measuring chair 20, the weak force variations (pressure changes) on the measuring sensors 2, 3, 6, 7, caused by the expansion and contraction of the lungs, the heart function, the blood circulation, body tremor, and the function of the digestive system of the patient, will cause changes in the signal level produced by the measuring sensors. By examining these variations in the signal level, it is possible to determine the condition of the patient's vital functions.

In the following, we shall discuss, with reference to FIGS. 3 and 4, some possible methods of analyzing the measuring signals 1−N in principle.

FIG. 3 shows a ballistocardiogram BCG which represents variations in the signal level caused by the patient's cardiac function, measured outside the body in the above-described manner by using the measuring chair 20 according to the invention.

Research on BCG measurements, as such, has been carried out from the 1940's to about the middle 1970's, but the research has had slow progress, because the apparatuses suitable for measuring BCG signals were, at that time, relatively complex and expensive mechanical constructions. Instead of the BCG measurement, the electrocardiogram (ECG), which is widely used at present, has been significantly easier to implement in the technical respect. However, the measuring chair 20 according to the invention eliminates this limitation, as it is particularly suitable for BCG measurements.

FIG. 3 shows the dominant mode of a BCG signal in a situation in which the signal is measured under the patient by measuring sensors placed in the seat part 17 of the measuring chair 20. In the Figure, the different points specific to the BCG signal are indicated with the letters F, G, H, I, J, K, L, M, and N, as is common practice in the field. The Figure also shows, with a dotted line, the moment of time when a so-called R-peak occurs in the electrocardiogram.

The span K-L in the BCG signal represents the condition of the peripheral blood circulation in the patient's body. The amplitude I-J, in turn, reflects the maximum force of the left chamber of the heart during a contraction. The amplitude I-J can thus indicate latent cardiac symptoms, early abnormalities and certain cardiac diseases, such as diseases of the aortic valve and coronary artery disease. Furthermore, the amplitude I-J can be used for the monitoring of the treatment of cardiac insufficiency or, for example, for the evaluation of the result of a bypass operation and for the correct dosage of medication effective on cardiac function. In this way, by controlling the dosage of the medication, a level is sought, at which the pumping capacity of the heart no longer responds significantly to an increase in the medication. The amplitude I-J has been utilized for the evaluation of the patient's life-time. According to some studies, a dramatic increase in the average amplitude I-J increases the number of cardiac arrhythmias at a probability of almost 50%.

The variable which best describes the patient's state of health is probably the I-J amplitude of the BCG signal, but also some other features of the BCG waveform correlate with the condition of the cardiovascular system of the patient under examination. The I-J amplitude is proportional to the condition of the heart in such a way that the higher said amplitude, the better the condition in which the patient's heart probably is. In this way, for example the effect of fitness training on the patient's health has been observed by measuring the BCG signal from a patient under examination before starting fitness training, and three and six months after starting fitness training, with a distinct increase in the I-J amplitude.

In addition to the above-described quantitative values, the BCG signal can also be analyzed qualitatively. In the qualitative classification of the BCG signal, the Starr classification, known as such, has often been applied. It is based on the comparison of the BCG signal measured from a patient with a BCG dominant mode of good quality and primarily on the recurrence of successive waveforms of good quality. The more waveforms deviating from the dominant mode are produced by heartbeats, the poorer the condition in which the heart is. If the dominant mode is only hardly, if at all, detectable here are there in the patient's BCG signal, the lifetime prognosis for the patient is not very good. Also these variables can be examined in a good signal analysis by means of the measuring chair according to the invention.

The BCG signal can also be used for monitoring the effects of medicaments and other substances in the body, for example by measuring tremor caused by the medicament in the person to be examined.

A reduction in the interval I-K and/or the amplitude I-K may occur in situations, in which the resistance of the blood vessels in the body or the stiffness of the wall of the aorta is increased. This may indicate the effect of increased blood pressure in the signal.

Diagnoses made by using the BCG signal are best in the setting up of short-term and long-term prognoses. Many persons older than 40 years, having no evidence of a cardiac disease, exhibit an abnormal BCG. This phenomenon has been utilized to detect coronary artery disease with little symptoms, and it has been used for the prognosis of the risk of acute cardiac thrombosis.

The invention is not limited solely to the measurement and analysis of signals from the patient's cardiac function. In addition to the cardiac function, the signals to be measured by means of the measuring chair 20 can also be used to detect variations in the signal level caused by respiration. Because these signal variations normally occur in a lower frequency range, the phenomena relating to the respiration can be separated from the phenomena relating to the cardiac function by means of a low-pass filter (and the cardiac function from the respiration by means of a high-pass filter). The examination of respiratory signal components may reveal, in addition to the respiratory frequency, various deficiencies in pulmonary function, such as obstructive ventilatory defect.

A variation in the amplitude of the respiratory signal element of the BCG signal may be used to detect latent coronary artery disease and possibly to give a prognosis on the lifetime. The BCG signal reflects the effect of the sympathetic and parasympathetic nervous systems on the contraction of the myocardium. The systolic main component of the BCG signal increases by the effect of inhalation and decreases during exhalation. The use of the inhalatory muscles during restrictive respiration may change the mechanics of the respiration and affect the BCG signal. Consequently, the BCG signal can also be used to detect diseases of the respiratory organs. In patients with asthma, with impaired pulmonary function, the failure in the pulmonary exhalation is manifested as flutter in the BCG signal. An obstruction in the airways is characterized by a variation in the amplitude of the BCG signal, and its magnitude indicates the degree of seriousness of the obstruction in the airways.

Variations caused by respiration in the BCG signal have been found to increase in pathological conditions, such as cardiac convulsion (angina of effort), cardiac failure and serious hypertension. Variations caused by respiration in the BCG signal are significantly greater in patients with a cardiac disease.

Furthermore, FIG. 4 shows examples of ballistocardiographic signals measured of testees. The upper graph CH2 in FIG. 4 was measured behind the patient's back by measuring sensors placed in the backrest 16 of the measuring chair 20, and the lower graph CH1 was measured under the patient by measuring sensors placed in the seat part 17.

The most significant advantage of the present invention is that no measuring sensors need to be attached to the person to be examined, wherein the measuring does not require special expertise of the person taking the measurements. Consequently, the person taking the measurements can be the person to be examined him/herself, or another person without particular medical training. Naturally, the measurements can also be taken by a professional in a health care organization or another corresponding organization.

The measurements are quick and easy to take, and do not require that the patient is particularly undressed. The measuring can also be started automatically when the patient is seated in the measuring chair 20. By means of the invention, the measurements of the patient's vital functions can also be taken in such a way that the person to be examined is not at all aware of the measuring situation. In this way, it is possible to avoid changes caused by a state of tension or other corresponding states in the vital functions which may lead to incorrect diagnoses. Also in such applications in which the person is aware of sitting in a measuring chair, the conventional appearance of the measuring chair reduces the probability that the measurement results are distorted because of the patient's anxiety.

When the apparatus according to the invention is used at home, measurements can be taken even several times a day, thanks to the easy measurement. Results are obtained from the measurement even though the person to be examined is simultaneously discussing with other persons present or, for example, is reading a paper at a table during the measurement.

As already mentioned above, the signal produced by the measuring sensors 2, 3, 6, 7 can be transmitted to a device for amplifying, analyzing or presenting it numerically or graphically, in a number of ways. The transmission can be implemented either through wires and connectors, or in a wireless manner. The measuring chair 20 may also comprise, entirely or partly, a device for amplifying, analyzing, or presenting the signal numerically, in text form, by means of a sound, or graphically. Such an implementation is probable, for example, in an automatic machine version for entertainment, in which the whole system is preferably provided in a compact unit.

There is a large variety of alternatives for the analysis of signals to be obtained from the measuring sensors 2, 3, 6, 7. By the analog technology, the signal can be filtered in various ways, for example by filtering out 50/60 Hz network noise, low-frequency variation caused by respiration, etc., in the preamplifier 21. Various intensities and time delays which are typical for signal waveforms and which, on the basis of prior art or other research, are known to correlate with a vital function or the condition of an organ, can also be measured from the signal. By digital signal processing, the same aspects can be implemented as by the analog technique, but the digital signal processing also provides other, more sophisticated filtering and analysis methods. For example, the signal can be averaged in relation to a typical signal waveform, either as linear combination of samples to be averaged or by a non-linear method based on, for example, order statistics, or as a combination of these. For example, by using autocorrelation, the pulse of the person to be examined can be computed from the signal obtained from the EMFi sensor. By digital signal processing, it is also easier to implement adaptive signal processing to adaptively reduce interference, for example by subtracting a signal obtained from a separate source of interference from the signal to be measured.

In addition to the time domain, the signals can also be analyzed in the frequency domain, for example by means of the Fourier transform, wavelet transform, or another transform, as well as by means of auto-regressive (AR) or autoregressive moving average (ARMA) models and by means of filter banks. By means of the transforms, it is possible to look for specific marks (signatures) in the signals, which signatures are known to correlate with some abnormal conditions. Such a method could be used, for example, in the comparison of the measurement result with reference material collected from normal persons and persons with various diseases.

In general, the signal processing method to be used in the apparatus can be either universal or particular signal analysis methods developed for the apparatus according to the invention. The signal processing methods can be implemented either by software, using a processor or the like designed for signal processing, or by using a special circuit designed for this method.

The measurement and analysis results can be presented either by a display device, such as a computer screen, a television, various flat display techniques, etc., or the results can also be printed on paper or another conventional printing material. The device for making the analysis can also give the result by means of sound. The sound may be either stored ready in the device, for example as a voice, wherein the device, on the basis of the analysis, selects one of the stored voices to be repeated (for example, “Your heart is in good condition and corresponds to the heart of a 25-year-old person”). The sound to be repeated can also be a directly measured signal or a signal produced from it by various modifications. From such a sound, a health care professional can, after gathering experience from the device, directly conclude some aspects of the patient's health. In this situation, the device would thus be used like a specialized stethoscope.

Various modifications of the measuring chair 20 can be made for different uses. A chair made to look like quite an ordinary chair and thereby unnoticeable as a medical apparatus may be suitable, for example, for the use of health care organizations to avoid the anxiety of the patient in a measuring situation. Such a chair could also be suitable for households, where it could easily fit in style with the other furniture at home. For home use, also another model could be suitable, equipped with the necessary displaying and/or printing facilities. The measuring chair 20 can thus be constructed to operate either as an independent, stand-alone apparatus, or to be used in connection with a separate computer device. In an automatic machine version for entertainment or pharmacy use, the measuring chair could typically comprise the display and/or printing facility, but it could also be connected, by conductive wires or in a wireless manner, to the computer of the organization holding the device, through which the display and printing operations are arranged.

1. An apparatus for measuring vital functions relating to the health of a patient, the device comprising at least a measuring chair (20) or a corresponding means suitable for sitting, which measuring chair further comprises one or more measuring sensors (2, 3, 6, 7) for measuring one or more vital functions of the patient in a non-invasive manner from the outside of the patient's body, characterized in that said one or more measuring sensors (2, 3, 6, 7) are placed in the structures of the measuring chair (20) in a substantially unnoticeable manner, and that the measuring chair (20) is designed to resemble an ordinary chair intended for non-medical use, or a corresponding furniture-like means suitable for sitting.

2. The apparatus according to claim 1, characterized in that said measuring sensors (2, 3, 6, 7) are structurally thin, preferably film-like sensors which are placed in the space between the frame (15) and the surface upholstery (13) of the measuring chair (20).

3. The apparatus according to claim 1, characterized in that at least some of said measuring sensors (2, 3, 6, 7) are implemented by using electromechanical film EMFi®.

4. The apparatus according to any of the preceding claims, characterized in that at least some of the measuring sensors (2, 3, 6, 7) are placed in the seat part (17) and/or the backrest (16) and/or the armrests (18) of the measuring chair (20).

5. The apparatus according to any of the preceding claims, characterized in that at least some of the measuring sensors (2, 3, 6, 7) are arranged to measure vital functions relating to the right and left sides of the patient's body separately.

6. The apparatus according to any of the preceding claims, characterized in that said measuring chair (20) or the like comprises at least
one or more first measuring sensors (2, 3),
cushion (4, 5) or a corresponding structure for attenuating vibrations, placed on said one or more first measuring sensors (2, 3),
one or more second measuring sensors (6, 7) installed on said cushion (4, 5) and preferably directly underneath the surface upholstery (13) against the person seated in the measuring chair (20), and
means for eliminating the common-mode signal recorded by said one or more first measuring sensors (2, 3) from the signal recorded by said one or more second measuring signals (6, 7).

7. The apparatus according to any of the preceding claims, characterized in that said measuring chair (20) also comprises one or more reference sensors (14) for measuring vibrations transmitted from the environment and for eliminating their effect from the signals of the measuring sensors (2, 3, 6, 7).

8. The apparatus according to any of the preceding claims, characterized in that the apparatus also comprises means for subjecting the signals recorded by the measuring sensors (2, 3, 6, 7) and/or the reference sensors (14) to one or more operations to be listed below: amplification (21), analog-to-digital conversion (22), filtering (21, 23), analysis (24), presentation (25), storage.

9. The apparatus according to claim 8, characterized in that said means are arranged to perform at least some of the operations listed in the claim by means of digital signal processing.

10. The apparatus according to claim 8 or 9, characterized in that said means are placed in whole or in part in the structures of the measuring chair (20).

11. The apparatus according to claim 8 or 9, characterized in that said means are placed, in whole or in part, in one or more electronic, signal processing, or data processing units separate from the measuring chair (20).

12. The apparatus according to claim 11, characterized in that the data transmission between the measuring chair (20) and said one or more separate units is implemented by wireless data transmission, preferably by using the Bluetooth data transmission technique.

13. The apparatus according to any of the preceding claims, characterized in that the apparatus is arranged to take ballistocardiographic measurements.

14. The apparatus according to any of the preceding claims, characterized in that the apparatus is arranged to take measurements relating to pulmonary functions.

15. The apparatus according to any of the preceding claims, characterized in that the apparatus is arranged to take measurements relating to the functions of the digestive system.

16. The apparatus according to any of the preceding claims, characterized in that the apparatus is arranged to measure effects caused by a medicament or by other treatment of the patient in the patient's body.
